(19) United States
(12) Patent Application Publication
(10) Pub. No.: US 2020/0352867 A1
(43) Pub. Date: Nov. 12, 2020

(54) PHARMACEUTICAL FORMULATION FOR A SOLID DOSAGE FORM OF OPIOID RECEPTOR ANTAGONISTS

(71) Applicant: TAIWANJ PHARMACEUTICALS CO., LTD., Zhubei City, Hsinchu County (TW)

(72) Inventors: Syaulan S. YANG, Zhubei City (TW); Eric Yueh-Lang SHEU, Clayton, CA (US); Ying-Chu SHIH, Zhubei City (TW)

(73) Assignee: TAIWANJ PHARMACEUTICALS CO., LTD., Zhubei City, Hsinchu County (TW)

(21) Appl. No.: 16/966,645

(22) PCT Filed: Feb. 1, 2019
(86) PCT No.: PCT/US2019/016264

§ 371 Date: Jul. 31, 2020

Related U.S. Application Data
(60) Provisional application No. 62/627,967, filed on Feb. 8, 2018.

(51) Int. Cl. A61K 9/28; A61K 31/485

(52) U.S. Cl. CPC A61K 9/2846; A61K 31/485

(57) ABSTRACT

The present disclosure provides a sustained release formulation of opioid receptor antagonists comprising a sustained release granule comprising at least one of the opioid receptor antagonist, at least one of pharmaceutical acceptable carrier, and a pH-dependent polymer, wherein the sustained release granule is coated with the pH-dependent polymer, and the opioid receptor antagonist is selected from the group consisting of Nalmefene, Naltrexone, or a salt thereof. The present disclosure further provides a method for preparing a sustained release formulation of opioid receptor antagonists comprising steps of: mixing at least one of the opioid receptor antagonist and at least one of pharmaceutical acceptable carrier to form a mixture; performing a wet granulation on the mixture with a pH-dependent polymer to form a sustained release granule; sieving the sustained release granule through a mesh screen to obtain a sieved sustained release granule; and compressing the sieved sustained release granule to obtain a sustained release (SR) formulation.

### BACKGROUND

Opioid receptor antagonist, also known as opioid antagonist, is a receptor antagonist that acts on opioid receptors.

Naltrexone and Nalmefene are commonly used opioid antagonist drugs. Naltrexone is a medication primarily used to manage alcohol dependence and opioid dependence while Nalmefene is a newer opioid antagonist which is structurally similar to Naltrexone but with potential pharmacological advantages for treating alcohol dependence. Naltrexone and Nalmefene are competitive antagonists that bind to the opioid receptors with higher affinity without activating the receptors. The block of the receptor prevents the body from responding to opioids and endorphins.

Both Naltrexone and Nalmefene are characterized by quite rapid absorption after oral administration. Since it has been reported that opioid receptors exist in the chemoreceptor trigger zone (CTZ) that interacts with the vomiting center (VC) in the brain, it is not difficult to rationalize the occurrence of opioid-like compounds causing central nervous system (CNS)-related adverse effects such as nausea and vomiting. In other words, oral administration of Naltrexone or Nalmefene may cause discomfort to patients due to their rapid absorption.

In terms of medical treatments including single or combo agent medications for long-term diseases comprising alcohol dependence or pain management, the aforementioned CNS-related adverse effects still need to be alleviated to improve the quality of life for patients.

Therefore, a novel pharmaceutical formulation for opioid receptor antagonists is needed.

### SUMMARY

An object of the present disclosure is to provide a sustained release formulation of opioid receptor antagonists comprising: a sustained release granule comprising at least one of the opioid receptor antagonist, at least one of pharmaceutical acceptable carrier, and a pH-dependent polymer, wherein the sustained release granule is coated with the pH-dependent polymer, and the opioid receptor antagonist is selected from the group consisting of Nalmefene, Naltrexone, or a salt thereof.

Another object of the present disclosure is to provide a sustained release solid form of opioid receptor antagonists comprising: a sustained release granule comprising at least one of the opioid receptor antagonist, at least one of pharmaceutical acceptable carrier, and a pH-dependent polymer; and an outer coating comprising the pH-dependent polymer, wherein the sustained release granule is coated with the pH-dependent polymer, and the opioid receptor antagonist is selected from the group consisting of Nalmefene, Naltrexone, or a salt thereof; and the sustained release solid form is surrounded with the outer coating.

Another object of the present disclosure is to provide a method for preparing a sustained release formulation of opioid receptor antagonists comprising steps of: mixing at least one of the opioid receptor antagonist and at least one of pharmaceutical acceptable carrier to form a mixture; performing a wet granulation on the mixture with a pH-dependent polymer to form a sustained release granule; sieving the sustained release granule through a mesh screen to obtain a sieved sustained release granule; and compressing the sieved sustained release granule to obtain a sustained release (SR) formulation, wherein the opioid receptor antagonist is selected from the group consisting of Nalmefene, Naltrexone, or a salt thereof.

### BRIEF DESCRIPTION OF DRAWINGS

The detailed description of the present disclosure will be made with reference to the accompanying drawing, where like numerals designate corresponding parts of the figures. The drawings are meant to be generally illustrative of various examples of the present disclosure, but are merely example and are not meant to be limiting the scope of the present disclosure.

FIG. 1 shows average (6 tablets) Nalmefene immediate release (NMF-IR) and Nalmefene sustained release NMF-SR) dissolution profiles in pH=6.8 buffer media using USP apparatus 2 method (USP<711>) in one embodiment of the present disclosure.

FIG. 2 shows average (6 tablets) Naltrexone immediate release (NTX-IR) and Naltrexone sustained release (NTX-SR) dissolution profiles in pH=6.8 buffer media using USP apparatus 2 method (USP<711>) in one embodiment of the present disclosure.

FIG. 3 shows mean plasma concentration-time profiles of Nalmefene immediate release tablet (NMF-IR) and the Nalmefene Sustained Release Tablet (NMF-SR), per oral (P.O.), in rats, in one embodiment of the present disclosure.

FIG. 4 shows average (6 tablets) Naltrexone immediate release (NTX-IR) and Naltrexone sustained release (further modified formulation) (NTX-SR2) dissolution profiles in pH=6.8 buffer media using USP apparatus 2 method (USP<711>) in one embodiment of the present disclosure.

FIG. 5 shows mean plasma concentration-time profiles of Naltrexone immediate release tablet (NTX-IR) and the Naltrexone Sustained Release (Further Modified Formulation) Tablet (NTX-SR2), per oral (P.O.), in dogs, in one embodiment of the present disclosure,

FIG. 6 shows Nalmefene sustained release SR3S (NMF-SR3S) and Nalmefene sustained release SR3L (NMF-SR3L) dissolution profiles in pH=6.8 buffer media using LISP apparatus 2 method (USP<711>) in one embodiment of the present disclosure.

FIG. 7 shows Naltrexone sustained release SR3S (NTX-SR3S) and Naltrexone sustained release SR3L (NTX-SR3L) dissolution profiles in pH=6.8 buffer media using USP apparatus 2 method (USP<711>) in one embodiment of the present disclosure.

### DETAILED DESCRIPTION OF THE INVENTION

The foregoing and other aspects of the present disclosure will now be described in more detail with aspect to other embodiments described herein. It should be appreciated that the present disclosure can be embodied in different forms and should not be construed as limited to the embodiments set forth herein. Rather, these embodiments are provided so that the present disclosure will be thorough and complete, and will fully convey the scope of the present disclosure to those skilled in the art.

The terminology used in the description of the present disclosure herein is for the purpose of describing particular embodiments only and is not intended to be limiting of the present disclosure. Unless otherwise defined, all technical and scientific terms used herein have the same meaning as commonly understood by one of ordinary skill in the art to which the present disclosure belongs. As used in the description of the present disclosure and the appended claims, the singular forms “a”, “an” and “the” are intended to include the plural forms as well, unless the context clearly indicates otherwise.

Unless otherwise indicated, the term “opioid” as used herein refers to compounds that exhibit opium or morphine-like properties, including agonist and antagonist activity wherein such compounds can interact with stereospecific and saturable binding sites in the brain and other tissues. The “opioid-like” as used herein refers to compounds that are similar in structure and/or pharmacological profile to known opioid compounds.

Unless otherwise indicated, the term “pharmaceutically acceptable” component (such as a salt, carrier, excipient or diluent) as used herein refers that the compound or composition is suitable for administration to a subject to achieve the treatments described herein, without unduly deleterious side effects in light of the severity of the disease and necessity of the treatment.

Unless otherwise indicated, the term “therapeutically effective amount” as used herein refers to an amount necessary to prevent, delay or reduce the severity of the condition of interest and also includes an amount necessary to enhance normal physiological functioning.

Unless otherwise indicated, the term “pharmaceutically acceptable carrier” as used herein refers to a carrier, whether diluent or excipient, that is compatible with the other ingredients of a formulation and not deleterious to recipient thereof. A usable pharmaceutically acceptable carrier are disclosed in various references including Handbook of Pharmaceuticals Excipients edited by Raymond C Rowe, Paul J Sheskey, and Marrian E Quinn. In an unlimited embodiment, the pharmaceutically acceptable carrier can be selected from the group consisting of inert diluents, dispersing and/or granulating agents, surface active agents and/or emulsifiers, disintegrating agents, binding agents, preservatives, buffering agents, lubricating agents, and/or oils. The compositions optionally further comprise at least one of additional biologically active compounds or agents.

Unless otherwise indicated, the term “pH-dependent polymer” as used herein refers to a broad family of polymers that substantially dissolve only above a specific trigger pH, and these polymers enable the pharmaceutical formulation to target specific areas of the intestine. The pH-dependent polymer is especially helpful for therapies that rely on targeted drug release in the high-pH colon area, i.e. for the local treatment of intestinal disorders such as Crohn's disease, ulcerative colitis or intestinal cancer.

An object of the present disclosure is to provide a sustained release formulation of opioid receptor antagonists comprising: a sustained release granule comprising at least one of the opioid receptor antagonist, at least one of pharmaceutical acceptable carrier, and a pH-dependent polymer, wherein the sustained release granule is coated with the pH-dependent polymer, and the opioid receptor antagonist is selected from the group consisting of Nalmefene, Naltrexone, or a salt thereof.

In some embodiments, the opioid receptor antagonists useful in the present disclosure include, for example and without limitation, naltrexone, nalmefene, naloxone, naltrindole, nalorphine, nalbuphene, normorphine, norpipanone, derivatives and analogs thereof. In some embodiments, the opioid receptor antagonist of the present disclosure includes naltrexone or nalmefene.

In some embodiments, Nalmefene or Naltrexone in the form of salt in the present disclosure may be in the form of an anhydrous salt, a mono-, di-, or multiple-hydrated salt or a mixture thereof, but it is not limited thereto. In some embodiments, Nalmefene or Naltrexone in the form of salt in the present disclosure may comprise, but is not limited to an inorganic acid salt of Nalmefene or Naltrexone and an organic acid salt of Nalmefene or Naltrexone, etc. Examples of inorganic acid for the inorganic acid salt ay comprise hydrochloric acid, hydrobromic acid, sulfuric acid, phosphoric acid, phosphorous acid and nitric acid, but they are not limited thereto. Moreover, examples of the organic acid for the organic acid salt may comprise acetic acid, malic acid, tartaric acid, formic acid, oxalic acid, lactic acid, citric acid, fumaric acid, cinnamic acid, salicylic acid, propionic acid, methanesulfonic acid, p-toluenesulfonic acid, ascorbic acid, gluconic acid and benzoic acid, but they are not limited thereto.

In some embodiments, Nalmefene or Naltrexone in the form of salt of the present disclosure may be Nalmefene or Naltrexone in the form of hydrochloride, hydrochloride dihydrate or a mixture thereof, in the pharmaceutical formulation of the present disclosure the Nalmefene or Naltrexone in the form of hydrochloride, hydrochloride dihydrate or a mixture thereof may be in amount of about 1-75 wt %. In one specific embodiment, in the pharmaceutical formulation of the present disclosure mentioned above, the Nalmefene or Naltrexone in the form of salt may be Nalmefene hydrochloride and it may be in amount of about 1-75 wt %, such as 5-35 wt %, or 10-29.5 wt %. In another specific embodiment, in the pharmaceutical formulation of the present disclosure, the Nalmefene or Naltrexone in the form of salt may be Naltrexone hydrochloride and/or hydrochloride dihydrate and it may be in amount of about 1-75 wt %, such as 5-35 wt %, or 10-29.5 wt %.

In some embodiments, the pH-dependent polymer of the present disclosure may comprises at least one acrylic polymer. The acrylic polymer may be cationic, anionic, or non-ionic polymers and may be based upon monomers of acrylates and/or methacrylates, for example, the polymers are formed of methacrylic acid or methacrylic acid esters. In one embodiment, the acrylic polymer is an anionic polymer. In some embodiments, the pH-dependent polymer of the present disclosure is selected from the group consisting of acrylic acid and methacrylic acid copolymers, methacrylic ester copolymers, ethoxyethyl methacrylate copolymers, methacrylicacylic acid copolymers, amino alkyl methacrylate copolymers and ammonioalkyl methacrylate copolymers, but it is not limited thereto. In some embodiments, the pH-dependent polymer of the present disclosure may be an anionic copolymers based on methacrylic acid and methyl methacrylate. In one preferred embodiment, the pH-dependent polymer is methacrylic ester copolymers.

In some embodiments, the pH-dependent polymer of the present disclosure may only dissolve above a specific trigger pH value. For example, the pH-dependent polymer of the present disclosure may have a dissolution above pH 5.0, pH 6.0, or pH 7.0. In one embodiment, the pH-dependent polymer of the present disclosure has a dissolution above pH 6.0. In one preferred embodiment, the pH-dependent polymer of the present disclosure has a dissolution above pH 7.0.

In some embodiments, a ratio of free carboxyl groups to ester groups of the pH-dependent polymer of the present disclosure is in a range from about 1:1 to about 1:2. In one embodiment, a ratio of free carboxyl groups to ester groups of the pH-dependent polymer of the present disclosure is about 1:1. In one preferred embodiment, a ratio of free carboxyl groups to ester groups of the pH-dependent polymer of the present disclosure is about 1:2.

In some embodiments, preferably, the pH-dependent polymer of the present disclosure is a methacrylic acid copolymer which is a commercially available from EUDRAGIT® polymers. EUDRAGIT® polymers are available in a wide range of different concentrations and physical forms, including aqueous solutions, aqueous dispersion, organic solutions, and solid substances. The pharmaceutical properties of the polymers are determined by the chemical properties of their functional groups. For example, EUDRAGIT® L, S, FS and E polymers have acidic or alkaline groups that are pH-dependent. Enteric EUDRAGIT® coatings provide protection against release of the GCC agonist in the stomach and enable controlled release in the intestine. In some embodiments, anionic EUDRAGIT® grades containing carboxyl groups are mixed with each other to provide pH-dependent release of the GCRA peptide and/or analogs. in some embodiments, EUDRAGIT® L and S grades are used for enteric coatings. In one embodiment, a ratio of the free carboxyl groups to the ester groups for EUDRAGIT® L is about 1:1. In one embodiment, a ratio of the free carboxyl groups to the ester groups for EUDRAGIT® S is about 1:2. The various EUDRAGIT® polymers are further described in international pharmacopeias such as Ph.Eur., USP/NF, DMF and JPE. Such a polymer is commercially available as EUDRAGIT® L, 100-55, EUDRAGIT® L 30D-55, EUDRAGIT® L, or EUDRAGIT® S 100 (commercially available from Rohm Pharma GmbH, Weiterstat, Germany).

In some embodiments, the pH-dependent polymer is selected from the group of methacrylic acid copolymers, preferably EUDRAGIT® S, and most preferably EUDRAGIT® S100. The preferred concentration is 10 wt % of the total weight of the dosage form, preferably 4-7 wt %.

In some embodiments, the sustained release granule of the present disclosure may comprise a wide variety of pharmaceutically acceptable carriers including binders, lubricants, anti-aggregating agents, disintegrants, dispersing and/or granulating agents, surface active agents and/or emulsifiers, preservatives, buffering agents, lubricating agents, and/or oils. In some embodiments, the pharmaceutical acceptable carrier of the present disclosure is selected from the group consisting of binders, lubricants, anti-aggregating agents, and disintegrants.

In some embodiments, for example, the binders of the present disclosure can include, but are not limited to, one or more of sugars such as glucose, lactose, sucrose; sugar alcohols such as xylitol, sorbitol or mannitol; polysaccharides such as starches, cellulose or derivatives of cellulose such as hydroxypropyl cellulose (HPC) or modified cellulose such as microcrystalline cellulose; synthetic polymers such as polyvinylpyrrolidone (PVP) or polyethylene glycol (PEG). In one preferred embodiment, the binders of the present disclosure include hydroxypropyl cellulose (HPC), mannitol, microcrystalline cellulose, or polyvinylpyrrolidone (PVP).

In some embodiments, the derivative of cellulose of the present disclosure may include, but is not limited to, hydroxypropyl celluloses (HPC), hydroxypropylmethylcellulose (HPMC) hydroxyethylcellulose (HEC), methylcellulose (MC), cellulose acetate, sodium carboxymethylcellulose, calcium salt of carboxymethylcellulose, ethylcellulose, etc. In one preferred embodiment, the derivative of cellulose of the present disclosure may be hydroxypropyl cellulose (HPC). The hydroxypropyl cellulose (HPC) may be in amount of 2-50 wt %, such as 5-15 wt %, or 10.5-14.5 wt % by weight of the dosage form.

In some embodiments, the mannitol of the present disclosure may be in amount of 2-50 wt %, such as 5-15 wt %, or 10.5-14.5 wt % by weight of the dosage form.

In some embodiments, the polyvinylpyrrolidone (PVP) of the present disclosure may have a viscosity k value between 5 and 180 measured at 1% aqueous solution, and have a pH value between 2 and 11 at 5% solid aqueous solution. Examples of the polyvinylpyrrolidone may include, but is not limited to, polyvinylpyrrolidone of K29-32 grade, K25 grade, K30 grade, or K40 grade. In one preferred embodiment, the polyvinylpyrrolidone (PVP) of the present disclosure may be polyvinylpyrrolidone of K29-32 grade and it may be in amount of 1-30 wt %, such as 5-15 wt %, or 10.5-13.9 wt % by weight of the dosage form.

In some embodiments, the microcrystalline cellulose of the present disclosure may be in amount of 3-60 wt %, such as 15-55 wt %, or 36.5-47.5 wt % by weight of the dosage form.

In some embodiments, for example, the lubricants of the present disclosure may include, but are not limited to, one or more of monoglyceride which may have waxy nature. The monoglyceride may have a melting point between 50° C. and 90° C., such as 50° C., 55° C., 60° C., 65° C., 70° C., 80° C., 83° C., 85° C., or 90° C., etc., but it is not limited thereto. Examples of the monoglyceride may include, but are not limited to, glycerol monomyristoleate, glycerol monopalmitoleate, glycerol monosapienate, glycerol monooleate, glycerol monoelaidate, glycerol monovaccenate, glycerol monolinoleate, glycerol monolinoelaidate, glycerol monolinolenate, glycerol monostearidonate, glycerol monoeicosenoate, glycerol monomeadate, glycerol monoarachidonate, glycerol monoeicosapentaenoate, glycerol monoerucate, glycerol monodocosahexaenoate, glycerol mononervonate, glyceryl dibehenate, glycerol behenate, glycerol dipalmitostearate, glycerol distearate, glycerol monolinoleate, and any combination thereof. In one preferred embodiment, the lubricant of the present disclosure may be glyceryl dibehenate. Moreover, the glyceryl dibehenate of the present disclosure may have a particle size between 0.5 μm and 500 μm, but it is not limited thereto. In one specific embodiment, the glyceryl dibehenate may be in amount of 0.01-10 wt %, such as 0.1-5 wt %, or 0.65-0.9 wt % by weight of the dosage form.

In some embodiments, for example, the lubricants of the present disclosure can include, but are not limited to, one or more of fumed silica. The fumed silica may have a specific surface area between 15 m²/g and 300 m²/g, but it is not limited thereto. Examples of the fumed silica may include, but are not limited to, hydrophobic fumed silica or hydrophilic fumed silica with water absorption ability. The hydrophilic fumed silica of the present disclosure may be a fumed silica treated with dimethyldichlorosilane, but is not limited thereto. The hydrophobic fumed silica of the present disclosure may have a SiO₂ content between 90 wt % and 99 wt %. The hydrophilic silica of the present disclosure with water absorption ability has a SiO₂ content between 10 wt % and 30 wt %. In one preferred embodiment, the lubricants of the present disclosure may be hydrophilic fumed silica. Moreover, the hydrophilic fumed silica of the present disclosure may be in amount of 0.01-10 wt %, such as 0.1-5 wt %, or 0.65-0.9 wt % by weight of the dosage form. A useful lubricant is a silica material, e.g., AEROSIL, which is a commercially available colloidal silica dioxide that is submicroscopic fumed silica with particle size of about 15 nm.

In some embodiments, in the sustained release formulation of opioid receptor antagonists of the present disclosure, the formulation is a sustained release form, such as sustained release tablets. The sustained release formulation of opioid receptor antagonists of the present disclosure is capable of providing a sustained release effect of Nalmefene or Naltrexone for the reduction of spike absorption in plasma concentrations.

In some embodiments, the sustained release formulation according to the present disclosure can be in any type of pharmaceutically acceptable dosage form comprising at least one therapeutically active agent and at least one pharmaceutical acceptable carrier. For example, the sustained release granule can be in the forms of liquids, semi-solids and solids, including pills, tablets, capsules and caplets. The preferred dosage forms for the sustained release granule of the present disclosure are tablets or capsules.

Another object of the present disclosure is to provide a sustained release solid form of opioid receptor antagonists comprising: a sustained release granule comprising at least one of the opioid receptor antagonist, at least one of pharmaceutical acceptable carrier, and a pH-dependent polymer; and an outer coating comprising the pH-dependent polymer, wherein the sustained release granule is coated with the pH-dependent polymer, and the opioid receptor antagonist is selected from the group consisting of Nalmefene, Naltrexone, or a salt thereof; and the sustained release solid form is surrounded with the outer coating.

In some embodiments, the sustained release formulation or the sustained release solid form of opioid receptor antagonists of the present disclosure may provide a longer delay of drug dissolution thereby allowing greater flexibility in designing sustained release profiles and further provide improved plasma levels wherein the maximum plasma concentration (Cₘₐₓ) can be substantially reduced without a concomitant reduction in AUC.

Another object of the present disclosure is to provide a method for preparing a sustained release (SR) formulation of opioid receptor antagonists. In some embodiments, the method for preparing a sustained release (SR) formulation of opioid receptor antagonists may comprise the following steps: mixing at least one of the opioid receptor antagonist and at least one of pharmaceutical acceptable carrier to form a mixture; performing a wet granulation on the mixture with a pH-dependent polymer to form a sustained release granule; sieving the sustained release granule through a mesh screen to obtain a sieved sustained release granule; and compressing the sieved sustained release granule to obtain a sustained release (SR) formulation, wherein the opioid receptor antagonist is selected from the group consisting of Nalmefene, Naltrexone, or a salt thereof.

In some embodiments, the pH-dependent polymer of the present disclosure may be dissolved in solvent to obtain a coating solution which be given clear to cloudy solutions. For example, the solvent of the present disclosure for dissolving the pH-dependent polymer include, but is not limited to, alcohols such as methanol, ethanol, propanol, isopropyl alcohol; ketones such as acetone; or pharmaceutical class 2 and 3 solvents or a combination thereof, etc. In one preferred embodiment, the solvent of the present disclosure for dissolving the pH-dependent polymer may be acetone. In some embodiments, the pH-dependent polymer of the present disclosure may be in amount of 1-15 wt % of the coating solution.

In some embodiments, the step of sieving the sustained release granule is performed through a mesh screen. In some embodiments, the mesh screen of the present disclosure has a mesh size between mesh #10 (1700 μm) and mesh #40 (380 μm), such as mesh #10 (1700 μm), or mesh π30 (550 μm).

In some embodiments, the step of compressing the sieved sustained release granule of the present disclosure may be performed by a compression force between 0.2 tons to 15 tons, such as 0,2 tons, 0.5 tons, I ton, 1.5 tons, 2 tons, 2.5 tons 10 tons, or 15 tons, In one preferred embodiment, the compression force of the present disclosure may be 2.5 tons.

In some embodiments, the method for preparing a sustained release (SR) formulation of opioid receptor antagonists of the present disclosure further comprises coating the sustained release (SR) formulation with a pH-dependent polymer. The pH-dependent polymer is the same as mentioned above.

### EXAMPLES

### Example 1

Nalmefene Sustained Release (SR) and Immediate Release (IR) Tablets.

Formulation for preparing both sustained release and immediate release tablets of Nalmefene are demonstrated in Table 1.

TABLE 1
Excipient of formulation for both Nalmefene Sustained
Release (SR) and Immediate Release (IR) Tablets

| | Actual | |
|---|---|---|
| | Formulation | Actual |
| | Weight (g) | Formulation % |
| Nalmefene HCl* (g) | 1.44016 | 26.47% |
| HPC (SSL) (g) | 0.61282 | 11.26% |
| Pearlitol (g) (mannitol) | 0.61282 | 11.26% |
| Prosolv 90 (g) | 2.08359 | 38.30% |
| (microcrystalline cellulose) | | |
| PVP K29/32 (g) | 0.61282 | 11.26% |
| Compritol 888 (glyceryl | 0.03922 | 0.72% |
| dibehenate) (g) | | |
| Aerosil R972 (g) | 0.03922 | 0.72% |
| (hydrophobic silica) | | |
| Total (g) | 5.44065 | 100% |

*The actual amount 1.44016 g of the weighed Nalmefene HCl salt contains 1.30096 g of Nalmefene (with a conversion factor of 1.107).

### Example 1.1

Preparation of Nalmefene Sustained Release (SR) Tablets (NMF-SR).

All ingredients listed in Table 1 including Nalmefene hydrochloride salt were mixed thoroughly and wet granulated with equal weight of a granulation solution containing 10 wt % of Eudragit (S-100) (methacrylic acid:methyl methacrylate (1:2) copolymer) in acetone. The granulated formulation was sieved through a #30 mesh screen to make granules. Following room temperature drying, magnesium stearate of 0.5 wt % was added to the granules before tablet press at 2.5 tons. The resulting tablets were then coated with a coating solution comprising with a 5.6 wt % of Eudragit (S-100) in mixed solvent diluents (2:1 by weight of isopropyl alcohol and acetone). The coating weight was approximately 2.5% of the uncoated tablet weight.

### Example 1.2

Preparation of Immediate Release (IR) Tablets (NMF-IR).

All ingredients listed in Table 1 including Nalmefene hydrochloride salt were blended thoroughly using an appropriate equipment (hand mixing was adequate for small quantity and V-blender for batch size over 0.5 kg). Magnesium stearate of no less than 0.01% and no more than 5% was added to the blended powder before tablet press with no less than 0.2 tons and no more than 15 tons of compression force. In this embodiment, the rat tablet was pressed at 2.5 tons and a weight of approximately 25 mg.

### Example 1.3

Release Profiles of Nalmefene Sustained Release (SR) Tablets and Nalmefene immediate Release (IR) Tablets.

The release profiles were measured according to USP <711> method (apparatus II). The media was a buffer solution with pH=6.8 to mimic the duodenum condition. The peddle speed was set at 50 rpm, temperature was controlled at 37° C., and the vessel volume was 600 mL. The sample collection time points were 0.5, 1, 2, 4, 6, 8, and 24 hours. The analysis was conducted using an Agilent 1100 Series HPLC following the guidelines of USP 37 Naltrexone Hydrochloride Assay procedure with a few modifications to the buffer gradient times. Below are the HPLC conditions, mobile phase preparation, and the gradient scheme.

Detection Wavelength: 210 nm

Column: Waters, C18, 100A, 4.6×100 mm, 3.5 μm

Column heater temperature: 40° C.

Mobile Phase: making buffer with 0.2% triethylamine and adjust pH to 5.0 with orthophosphoric acid. Mixing the buffer with acetonitrile at 85:15 volume ratio and degas through 0.45 μm membrane before use.

Flow rate: 0.7 mL/minute

Injection volume: 10 μL

Run Time: 30 minutes

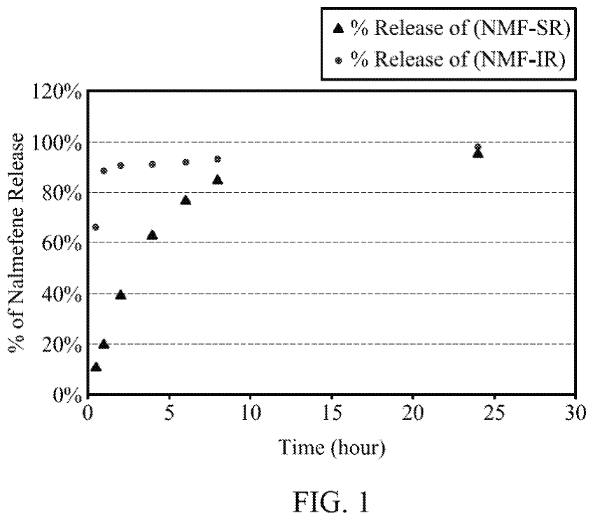

The average dissolution profiles (from 6 tablets used for rat pharmacokinetic study) for both Nalmefene sustained release (SR) and immediate release (IR) tablets are shown in FIG. 1.

### Example 2

Naltrexone Sustained Release (SR) and immediate Release (IR) Tablets.

Formulation for preparing both sustained release and immediate release tablets of Naltrexone are demonstrated in Table 2.

TABLE 2
Excipient of formulation for both Naltrexone Sustained
Release (SR) and Immediate Release (IR) Tablets

| | Actual | |
|---|---|---|
| | Formulation | Actual |
| | Weight (g) | Formulation % |
| Naltrexone HCl* (g) | 1.64026 | 29.08% |
| HPC (SSL) (g) | 0.61285 | 10.86% |
| Pearlitol (g) | 0.61285 | 10.86% |
| (mannitol) | | |
| Prosolv 90 (g) | 2.08368 | 36.94% |
| (microcrystalline cellulose) | | |
| PVP K29/32 (g) | 0.61285 | 10.86% |
| Compritol 888 (glyceryl | 0.03922 | 0.70% |
| dibehenate) (g) | | |
| Aerosil R972 (g) | 0.03922 | 0.70% |
| (hydrophobic silica) | | |
| Total (g) | 5.64093 | 100% |

*The actual amount 1.64026 g of the weighed Naltrexone HCl—2H₂O salt contains 1.35335 g of Naltrexone (with a conversion factor of 1.212).

### Example 2.1

Preparation of Naltrexone Sustained Release (SR) Tablets (NTX-SR).

All ingredients listed in Table 2. including Naltrexone hydrochloride, or hydrochloride dihydrate salt were mixed thoroughly and wet granulated with equal weight of a granulation solution containing 10 wt % of Eudragit (S-100) (methacrylic acid:methyl methacrylate (1:2) copolymer) in acetone. The granulated formulation was sieved through a #30 mesh screen to make granules. Following room temperature drying, 0.5 wt % of magnesium stearate was added to the granules before tablet press at 2.5 tons. The resulting tablets were then coated with a coating solution comprising with a 5.6 wt % of S-100 in mixed solvent diluents (2:1 by weight of isopropyl alcohol and acetone). The coating weight was approximately 2.5 wt % of the uncoated tablet weight.

### Example 2.2

Preparation of Naltrexone Immediate Release (IR) Tablets (NTX-IR).

All ingredients listed in Table 2 including Naltrexone hydrochloride, or hydrochloride dihydrate salt were blended thoroughly using appropriate equipment (hand mixing is adequate for small quantity and V-blender for batch size over 0.5 kg). Magnesium stearate of no less than 0.01% and no more than 5% was added to the blended powder before tablet press with no less than 0.2 tons and no more than 15 tons of compression force.

### Example 2.3

Release Profiles of Naltrexone Sustained Release (SR) Tablets and Naltrexone Immediate Release (IR) Tablets

The release profiles were measured according to USP <711> method (apparatus II). The media was a buffer solution with pH=6.8 to mimic the duodenum condition. The peddle speed was set at 50 rpm, temperature controlled at 37° C., and the vessel volume was 600 mL. The sample collection time points were 0.5, 1, 2, 4, 6, 8, and 24 hours. The analysis was conducted using an Agilent 1100 Series HPLC following the guidelines of USP 37 Naltrexone Hydrochloride Assay procedure with a few modifications to the buffer gradient times. Below are the HPLC conditions, mobile phase preparation, and the gradient scheme.

Detection Wavelength: 280 nm

Column: Phenomenex Luna 3 μm, C18, 3.9-mm×15-cm, L1 packing

Column heater temperature: 40° C.

Mobile Phase A: Dissolve 1.08 g of sodium 1-octanesulfonate and 23.8 g of sodium acetate with 800 mL of HPLC water and 200 mL of methanol. Add 1.0 mL of triethylamine and adjust the pH to 6.5 with glacial acetic acid. Filter through PVDF (Millicup®-HV (0.45 μm) or equivalent) with a vacuum.

Mobile Phase B: Dissolve 1.08 g of sodium 1-octanesulfonate and 23.8 g of sodium acetate with 600 mL of HPLC water and 400 mL of methanol. Add 1.0 mL of triethylamine and adjust the pH to 6.5 with glacial acetic acid. Filter through PVDF (Millicup®-HV (0.45 μm) or equivalent) with a vacuum.

Flow rate: 1.0 mL minute

Injection volume: 20 μL

Run Time: 30 minutes

TABLE 3
Gradient scheme for HPLC

| Time (minutes) | % Mobile Phase A | % Mobile Phase B |
|---|---|---|
| 0 | 100 | 0 |
| 20 | 0 | 100 |
| 21 | 100 | 0 |
| 30 | 100 | 0 |

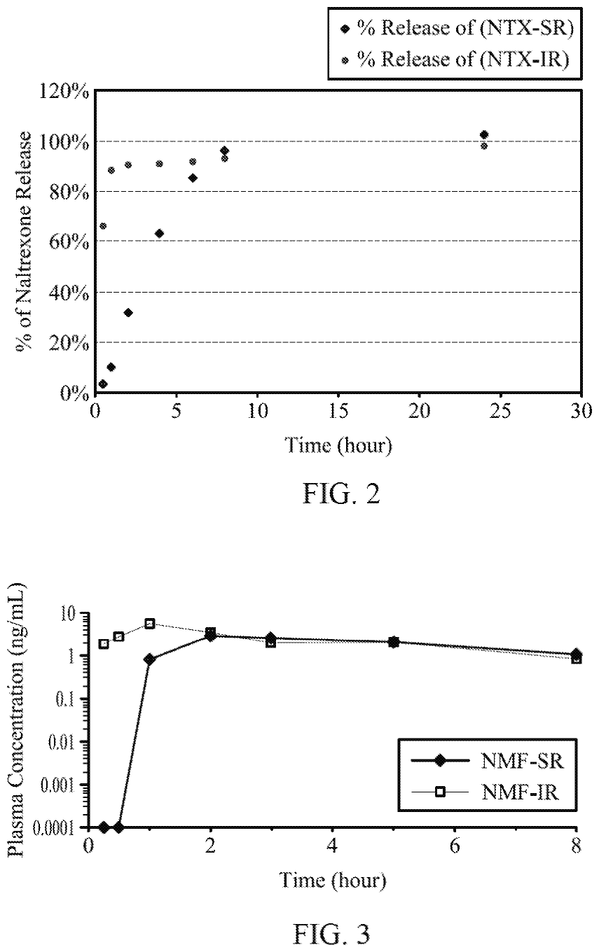

The average dissolution profiles (from 6 tablets used for rat pharmacokinetic study for both IR and SR Naltrexone tablets are shown in FIG. 2.

### Example 3

Pharmacokinetic Measurement of Nalmefene Immediate Release Tablet (NMF-IR) and Sustained Release Tablet (NMF-SR)

Animals

Male Sprague-Dawley (SD) rats weighing 180-250 g were provided by BioLasco Taiwan (under Charles River Laboratories License). Space allocation for three animals was 47×25×21 cm. All animals were maintained in a controlled temperature (20-24° C.) and humidity (30% -70%) environment with 12 hours light/dark cycles in laboratory. Free access to standard lab diet [MFG (Oriental Yeast Co., Ltd., Japan)] and autoclaved tap water were granted. All aspects of this work including housing, experimentation, and animal disposal were performed in general accordance with the “Guide for the Care and Use of Laboratory Animals: Eighth Edition” (National Academies Press, Washington, D.C., 2011) in the AAALAC-accredited laboratory animal facility. In addition, the animal care and use protocol were reviewed and approved by the IACUC at Pharmacology Discovery Services Taiwan, Ltd.

Chemicals

0.9% NaCl (Sing-Tong, Taiwan), and WFI (Tai-Yu, Taiwan).

Equipment

0-1000 g Electronic scale (Tanita Corporation, Japan), Animal cage (Allentown, USA), Centrifuge 5810R (Eppendrof, Germany), Disposal syringe (1 and 3 mL, Terumo Corporation, Japan), Forceps (Klappencker, Germany), Microcentrifuge tubes 1.5 mL click-cap (Treff AG, Switzerland), MiniCollect lithium heparin tube (Greiner Bio-One, Austria), Pipetman (# P200 Gilson, France), Pipette Tips (Costar, USA), and Stop watch (Casio, China).

Test Article Dosing & YK Sample Collection

The details of test article dosing and PK sample collection are illustrated in Table 4.

TABLE 4
Test Article Dosing & PK Sample Collection
Test Article Dosing and PK Sample Collection

| | |
|---|---|
| Test Article ID | NMF-SR, and NMF-IR |
| Target for Bioanalysis | Groups 1-2: Nalmefene; NMF |
| Route(s) of Administration: | PO |
| Dose Levels (mg/kg) | 30 mg/kg for a 200 g rat |
| Dose Volume | 1 tablet/rat (6 mg/tablet) |
| Proposed Formulation/Vehicle: | NA |
| Number of Animals per Dosing | 3 |
| Group: | |
| Total Number of Animals: | 6 |
| Total Number of Samples: | 48 plasma |
| Blood (PK Sample) | 15, 30, 60, 120, 180, 300, |
| Collection Time: | 480 and 1,440 minutes |
| Target Blood Sample | No less than 0.3 mL |
| Volume (mL): | by catheter |
| Preferred Anticoagulation: | Lithium heparin |
| Preferred Sample Storage: | −70° C. |

Plasma Sample Collection from Rats (Serial Sampling)

Blood aliquots (300-400 μL) were collected from jugular vein catheterized rats in tubes coated with lithium heparin, mixed gently, then kept on ice and centrifuged at 2,500×g for 15 minutes at 4° C., within 1 hour of collection. For control animals, blood was collected by cardiac puncture. The plasma was then harvested and kept frozen at −70° C. until further processing.

Quantitative Bioanalysis (Plasma)

The plasma samples were processed using acetonitrile precipitation and analyzed by LC-MS/MS. A plasma calibration curve was generated. Aliquots of drug-free plasma were spiked with the test compound at the specified concentration levels. The spiked plasma samples were processed together with the unknown plasma samples using the same procedure. The processed plasma samples were stored at −70° C. until the LC-MS/MS analysis, at which time peak areas were recorded, and the concentrations of the test compound in the unknown plasma samples were determined using the respective calibration curve, The reportable linear range of the assay was determined, along with the lower limit of quantitation (LLQ).

Pharmacokinetics

Plots of plasma concentration of compound versus time were constructed. The fundamental pharmacokinetic parameters of compound after PO dosing (AUCₗₐₛₜ, AUC_{INF}, T_{1/2}, Cl, V_{z}, Vₛₛ, Tₘₐₓ, and Cₘₐₓ) are obtained from the non-compartmental analysis (NCA) of the plasma data using WinNonlin.

The pharmacokinetic parameters of Nalmefene immediate release tablet (NMF-IR) and Nalmefene sustained release tablet (NMF-SR) were measured and the results are in Table 5.

TABLE 5
Pharmacokinetic Parameters of Nalmefene Immediate Release Tablet
(NMF-IR) and Nalmefene Sustained Release Tablet (NMF-SR)

| | |
|---|---|
| NMF-IR | NMF-SR |

| Tablet | Mean | SD | Mean | SD |
|---|---|---|---|---|
| Tₘₐₓ (hour) | 1.333 | 0.577 | 3.333 | 1.528 |
| Cₘₐₓ (ng/mL) | 5.30 | 2.05 | 2.84 | 0.58 |
| AUCₗₐₛₜ (h*ng/mL) | 17.22 | 1.64 | 12.83 | 2.65 |
| AUC_{Inf} (h* ng/mL) | 21.00 | 0.83 | 18.78 | 3.82 |
| AUC/D | 0.7 | 0.0 | 0.6 | 0.1 |
| (h*kg*ng/ml/mg) | | | | |
| AUC Extr (%) | 17.87 | 8.71 | 31.70 | 1.01 |
| MRT (hour) | 4.85 | 1.01 | 7.14 | 0.27 |

Based on the measured pharmacokinetic data, the mean plasma concentration-time profiles of the Nalmefene immediate release tablet (NMF-IR) and the Nalmefene sustained release tablet (NMF-SR) by oral administration in rats can be plotted as in FIG. 3.

It will be apparent to those skilled in the art that various modifications and variations can be made to the disclosed embodiments. It is intended that the specification and examples be considered as exemplary only, with the true scope of the disclosure being indicated by the following claims and their equivalents.

### Example 4

Naltrexone Sustained Release (Further Modified Formulation) (SR2) Tablets.

Further modified formulation for preparing sustained release tablets of Naltrexone are demonstrated in Table 6.

TABLE 6
Excipient of further modified formulation for
Naltrexone Sustained Release (SR2) Tablets

| | Actual | |
|---|---|---|
| | Formulation | Actual |
| | Weight (g) | Formulation % |
| Naltrexone HCl* (g) | 0.28221 | 10.27% |
| HPC (SSL) (g) | 0.37813 | 13.76% |
| Pearlitol 200 SD (g) | 0.37780 | 13.75% |
| (mannitol) | | |
| Prosolv 90 (g) | 1.28331 | 46.71% |
| (microcrystalline | | |
| cellulose) | | |
| PVP K29/32 (g) | 0.37755 | 13.74% |
| Compritol 888 | 0.02423 | 0.88% |
| (glyceryl | | |
| dibehenate) (g) | | |
| Aerosil R972 (g) | 0.02411 | 0.88% |
| (hydrophobic silica) | | |
| Total (g) | 2.74733 | 100% |

*The actual amount 0.28221 g of the weighed Naltrexone HCl anhydrous salt contains 0.25497 g of Naltrexone (with a conversion factor of 1.107).

### Example 4.1

Preparation of Naltrexone Sustained Release (Further Modified Formulation) (SR2) Tablets (NTX-SR2).

The preparation procedure is similar to the one described in Example 2.1 except with a coating solution comprising with a 14.67 wt % of S-100 in mixed solvent diluents (2:1 by weight of isopropyl alcohol and acetone). The total amount of 2.74733 g material was used to make about 15 tablets with 170-175 mg each to account for the S-100 content.

Example 4.2

Release Profile of Naltrexone Sustained Release (Further Modified Formulation) (SR2) Tablets (NTX-SR2).

The release profile was measured by using the same method as in Example 2.3.

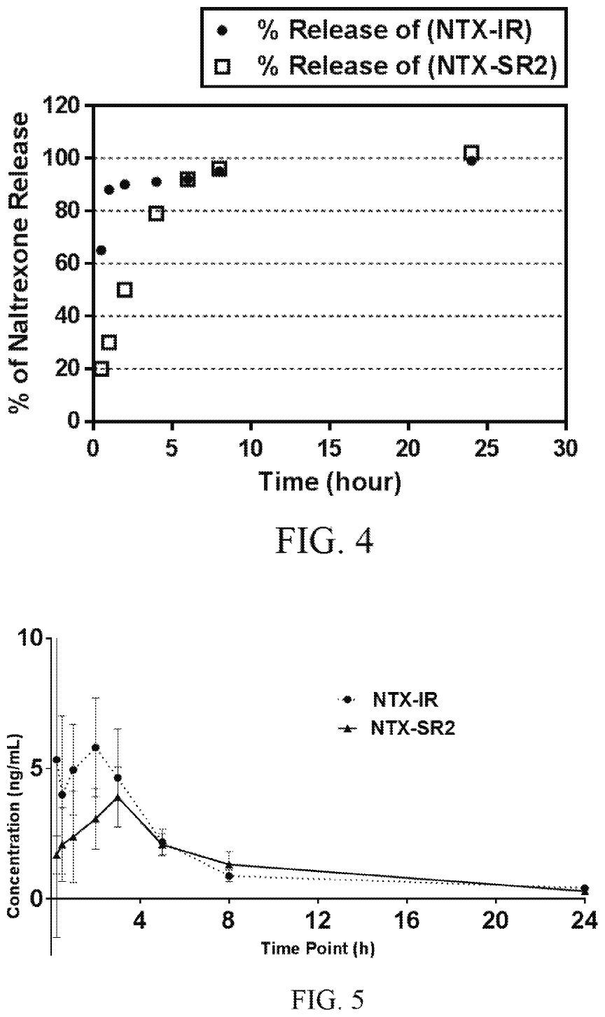

The average dissolution profiles for both IR and SR2 (further modified formulation) of Naltrexone tablets are shown in FIG. 4.

### Example 5

Pharmacokinetic Measurement of Naltrexone Immediate Release Tablet (NTX-IR) and Sustained Release (Further Modified Formulation) (SR2) Tablet (NTX-SR2)

Animals

The dog PK study was carried out after the assurance of no observable adverse events at the same dose in lower species.

The test animal description is listed in Table 7.

TABLE 7
Test Animal Description for the Dog PK Study

| | |
|---|---|
| Species: | Canis familiaris |
| Initial Age: | 1-6.5 yrs |
| Sex: | Male |
| Breed: | Beagle |
| Initial Body Weight: | ~8-14 kg |
| Source of Animals: | Marshall Bioresources, |
| | North Rose, New York |
| Identification | Animals were housed one per cage. |
| Method: | They were each assigned a study |
| | number and identified by ear tag |
| | and cage label. |
| Experimental Unit: | Individual animal |
| Replicates per | N = 3 per dose group per leg, |
| Treatment: | total 3 dogs (crossover) |
| Inclusion Criteria: | Animals were healthy at the start, |
| | of the trial. |
| Exclusion Criteria: | Any of the above inclusion criteria |
| | out of specification. |
| Blinding of Study: | The study was not blinded. |

Test Article Dosing & PK Sample Collection

The details of test article dosing and PK sample collection are illustrated in Table 8.

TABLE 8
Test Article Dosing & PK Sample Collection

| | | Dosing | | | Blood Sampling |
|---|---|---|---|---|---|
| Leg # | Test Article | Route | N= | Dose | Time Points |
| 1 | Naltrexone-IR | PO | 3 | 1 Tablet | Pre-dose, 15 min, 30 min, |
| | | | | | 1, 2, 3, 5, 8, and 24 |
| | | | | | hours post dose |

Minimum 7 Day Washout

| | | | | | |
|---|---|---|---|---|---|
| 2 | Naltrexone-SR2 | PO | 3 | 1 Tablet | Pre-dose, 15 min, 30 min, |
| | | | | | 1, 2, 3, 5, 8, and 24 |
| | | | | | hours post dose |

Blood Sample Site/Volume: Jugular vein, or other suitable vessel, ~1 mL
Type of Blood Tubes: K₂EDTA
Type of Sample: Plasma
Sample Storage and Shipment: ~−70° C.
Washout Interval: Minimum 7-day washout

Plasma Sample Collection from Dogs

Each blood sample was collected from the dog, jugular vein, or other suitable vessel via direct venipuncture, placed into a chilled tube containing K₂EDTA as the anticoagulant, and inverted several times to mix. Blood samples were kept on wet ice until centrifugation that was carried out at a temperature of 4° C., at 3,000×g, for 5 minutes. All samples were maintained chilled throughout processing. Plasma were collected, aliquoted into cluster tubes populated in a 96 well plate, and placed in a freezer set to maintain ˜−70° C. until analysis.

Quantitative Bioanalysis (Plasma)

Plasma samples were manually extracted via precipitation with acetonitrile in a 96-well plate, followed by LC-MS/MS analysis equipped with a Waters Acquity UPLC system connected with a Waters Xevo TQ-S Mass Spectrometer. Assay acceptance criteria of the analytical method was that at least 60% of the calibration standards must be within ÷20% of nominal, except at the LLOQ where ±25% is acceptable, in order for the analytical run to pass.

Pharmacokinetics

Pharmacokinetic parameters were calculated from the time course of the plasma concentration and determined with Phoenix WinNonlin (v8.0) software using a non-compartmental model. The maximum plasma concentration (Cₘₐₓ) and the time to reach maximum plasma concentration (tₘₐₓ) after PO dosing were observed from the data. The area under the time-concentration curve (AUC) was calculated using the linear trapezoidal rule with calculation to the last quantifiable data point (AUC₀₋ₗₐₛₜ), and with extrapolation to infinity (AUC_{∞}) if applicable. Plasma half-life (t_{1/2}) was calculated from 0.693/slope of the terminal elimination phase. Mean residence time, MRT, was calculated by dividing the area under the moment curve (AUMC) by the AUC. Clearance was calculated as Dose/AUC_{∞}. Volume of distribution=MRT_{∞}*Cl. Any samples below the limit of quantitation (0.125 ng/mL) were treated as zero for calculation of mean values.

The pharmacokinetic parameters of Naltrexone immediate release tablet (NTX-IR) and Naltrexone sustained release tablet (further modified formulation) (NTX-SR2) we measured and the results are in Table 9.

TABLE 9
Pharmacokinetic Parameters of Naltrexone Immediate Release
Tablet (NTX-IR) and Naltrexone Sustained Release Tablet
(Further Modified Formulation) (NTX-SR2)

| | |
|---|---|
| NTX-IR | NTX-SR2 |

| Tablet | Mean | SD | Mean | SD |
|---|---|---|---|---|
| Tₘₐₓ (hour) | 1.75 | 1.39 | 2.67 | 0.577 |
| Cₘₐₓ (ng/mL) | 7.90 | 4.79 | 4.11 | 1.18 |
| AUCₗₐₛₜ (h*ng/mL) | 36.5 | 8.65 | 32.1 | 2.82 |
| AUC_{Inf} (h*ng/mL) | 48.6 | NA | 35.1 | 2.24 |
| AUCₗₐₛₜ/D | 29.3 | 6.19 | 26.3 | 0.792 |
| (h*kg*ng/ml/mg) | | | | |
| MRT (hour) | 5.95 | 1.06 | 6.53 | 0.801 |

Based on the measured pharmacokinetic data, the mean plasma concentration-time profiles of the Naltrexone immediate release tablet (NTX-IR) and the Naltrexone sustained release (further modified formulation) tablet (NTX-SR2) by oral administration in dogs can be plotted as in FIG. 5.

### Example 6

Nalmefene Sustained Release SR3S (NMF-SR3S) and Nalmefene Sustained Release SR3L (NMF-SR3L) Tablets.

Formulations for preparing two of sustained release SRR3 and SR3L tablets of Nalmefene are demonstrated in Table 10.

TABLE 10
Excipient of formulation for two of sustained
release SRR3 and SR3L Tablets

| | Actual | |
|---|---|---|
| | Formulation | Actual |
| | Weight (g) | Formulation % |
| Nalmefene HCl* (g) | 1.30096 | 24.54% |
| HPC (SSL) (g) | 0.61282 | 11.56% |
| Pearlitol (g) | 0.61282 | 11.56% |
| (mannitol) | | |
| Prosolv 90 (g) | 2.08359 | 39.30% |
| (microcrystalline | | |
| cellulose) | | |
| PVP K29/32 (g) | 0.61282 | 11.56% |
| Compritol 888 | 0.03922 | 0.74% |
| (glyceryl | | |
| dibehenate) (g) | | |
| Aerosil R972 (g) | 0.03922 | 0.74% |
| (hydrophobic silica) | | |
| Total (g) | 5.3015 | 100% |

*The actual amount 1.44016 g of the weighed Nalmefene HCl salt contains 1.30096 g of Nalmefene (with a conversion factor of 1.107).

### Example 6.1

Preparation of Sustained Release SRR3 and SR3L of Nalmefene.

All ingredients listed in Table 10 including Nalmefene hydrochloride salt were mixed thoroughly and wet granulated with equal weight of a granulation solution containing 10 wt % of Eudragit (S-100) in acetone (methacrylic acid:methyl methacrylate (1:2) copolymer) for Nalmefene Sustained Release SR3S (NMF-SR3S) or 10 wt % of Eudragit (L-100) in acetone (methacrylic acid:methyl methacrylate (1:1) copolymer) for Nalmefene Sustained Release SR3L (NMF-SR3L). The granulated formulation was sieved through a #30 mesh screen to make granules. Following room temperature drying, magnesium stearate of 0.5 wt % was added to the granules before tablet press at 2.5 tons, The resulting tablets were then coated with a coating solution comprising with a 2 wt % of Eudragit (S-100) in mixed solvent diluents (2:1 by weight of isopropyl alcohol and acetone) for NMF-SR3S or with a 2 wt % of Eudragit (L-100) in mixed solvent diluents (2:1 by weight of isopropyl alcohol and acetone) for NMF-SR3L. cl Example 6.2

Release Profiles of Nalmefene Sustained Release SR3S (NMF-SR3S) Tablets and Nalmefene Sustained Release SR3L (NMF-SR3L)Tablets

The release profiles were measured by using the same method described in Example 1.3.

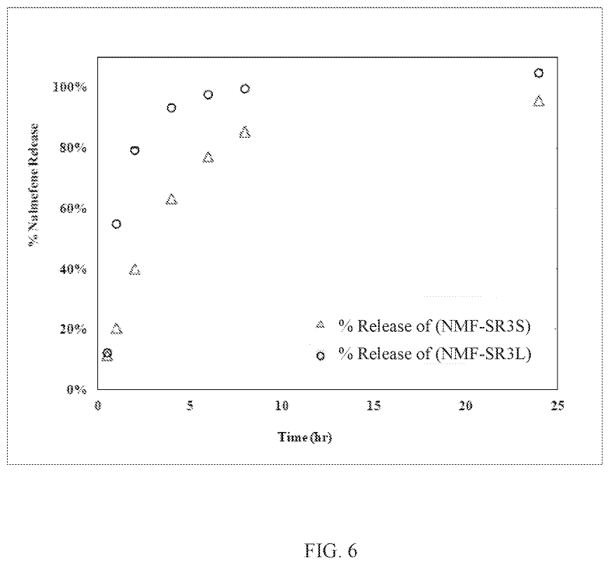

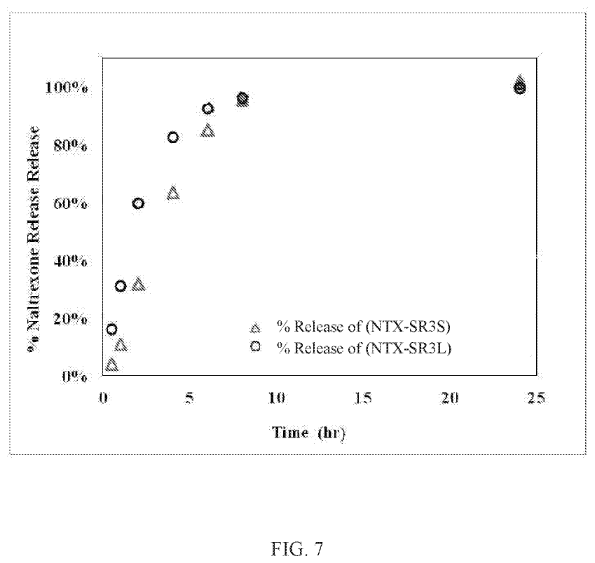

The average dissolution profiles for Nalmefene Sustained Release SR3S (NMF-SR3S) Tablets and Nalmefene Sustained Release SR3L (NMF-SR3L) tablets are shown in FIG. 6.

### Example 7

Naltrexone Sustained Release SR3S (NTX-SR3S) and Naltrexone Sustained Release SR3L (NTX-SR3L) Tablets.

Formulation for preparing two of sustained release SRR3 and SR3L tablets of Naltrexone are demonstrated in Table 11.

TABLE 11
Excipients of formulation for two of sustained
release SRR3 and SR3L Tablets

| | Actual | |
|---|---|---|
| | Formulation | Actual |
| | Weight (g) | Formulation % |
| Naltrexone HCl* (g) | 1.35335 | 25.28% |
| HPC (SSL) (g) | 0.61285 | 11.45% |
| Pearlitol (g) (mannitol) | 0.61285 | 11.45% |
| Prosolv 90 (g) (microcrystalline | 2.08368 | 39.92% |
| cellulose) | | |
| PVP K29/32 (g) | 0.61285 | 11.45% |
| Compritol 888 (glyceryl dibehenate) | 0.03922 | 0.73% |
| (g) | | |
| Aerosil R972 (g) (hydrophobic silica) | 0.03922 | 0.73% |
| Total (g) | 5.3540 | 100% |

*The actual amount 1.64026 g of the weighed Naltrexone HCl salt contains 1.35335 g of Naltrexone (with a conversion factor of 1.212).

### Example 7.1

Preparation of Sustained Release SRR3 and SR3L of Naltrexone.

All ingredients listed in Table 11 including Nalmefene hydrochloride salt were mixed thoroughly and wet granulated with equal weight of a granulation solution containing 10 wt % of Eudragit (S-100) in acetone (methacrylic acid:methyl methacrylate (1:2) copolymer) for Naltrexone Sustained Release SR3S (NTX-SR3S) or 10 wt % of Eudragit (L-100) in acetone (methacrylic acid:methyl methacrylate (1:1) copolymer) for Naltrexone Sustained Release SR3L (NTX-SR3L). The granulated formulation was sieved through a #30 mesh screen to make granules. Following room temperature drying, magnesium stearate of 0.5 wt % was added to the granules before tablet press at 2.5 tons. The resulting tablets were then coated with a coating solution comprising with a 2 wt % of Eudragit (S-100) in mixed solvent diluents (2:1 by weight of isopropyl alcohol and acetone) for NTX-SR3S or with a 2 wt % of Eudragit (L-100) in mixed solvent diluents (2:1 by weight of isopropyl alcohol and acetone) for NTX-SR3L.

### Example 7.2

Release Profiles of Naltrexone Sustained Release SR3S (NTX-SR3S) Tablets and Naltrexone Sustained Release SR3L (NTX-SR3L)Tablets

The release profiles were measured by using the same method described in Example 2.3.

The average dissolution profiles for Naltrexone Sustained Release SR3S (NTX-SR3S) Tablets and Naltrexone Sustained Release SR3L (NTX-SR3L) tablets are shown in FIG. 7.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A sustained release formulation of opioid receptor antagonists comprising:
a sustained release granule comprising at least one of the opioid receptor antagonist, at least one of pharmaceutical acceptable carrier, and a pH-dependent polymer,
wherein the sustained release granule is coated with the pH-dependent polymer, and the opioid receptor antagonist is selected from the group consisting of Nalmefene, Naltrexone, or a salt thereof.

2. The sustained release formulation of claim 1, wherein the opioid receptor antagonist is in the form of an anhydrous salt, a mono-, di-, or multiple-hydrated salt or a mixture thereof.

3. The sustained release formulation of claim 1, wherein the pH-dependent polymer has a dissolution above pH 6.0.

4. The sustained release formulation of claim 1, wherein the pH-dependent polymer is selected from the group consisting of acrylic acid and methacrylic acid copolymers, methacrylic ester copolymers, ethoxyethyl methacrylate copolymers, methacrylicacylic acid copolymers, amino alkyl methacrylate copolymers, and ammonioalkyl methacrylate copolymers.

5. The sustained release formulation of claim 1, wherein a ratio of free carboxyl groups to ester groups of the pH-dependent polymer is in a range from about 1:1 to about 1:2.

6. The sustained release formulation of claim 1, wherein the pharmaceutical acceptable carrier is selected from the group consisting of binders, lubricants, anti-aggregating agents, and disintegrants.

7. The sustained release formulation of claim 1, further comprises an outer coating surrounds the sustained release formulation.

8. The sustained release formulation of claim 7, wherein the outer coating comprises a pH-dependent polymer.

9. The sustained release formulation of claim 1, wherein the sustained release formulation is in a form of tablet, capsule, or caplet.

10. A sustained release solid form of opioid receptor antagonists comprising:
a sustained release granule comprising at least one of the opioid receptor antagonist, at least one of pharmaceutical acceptable carrier, and a p1-1-dependent polymer; and
an outer coating comprising the pH-dependent polymer,
wherein the sustained release granule is coated with the pH-dependent polymer, and the opioid receptor antagonist is selected from the group consisting of Nalmefene, Naltrexone, or a salt thereof and the sustained release solid form is surrounded with the outer coating.

11. The sustained release solid form of claim 10, wherein the opioid receptor antagonist is in the form of an anhydrous salt, a mono-, di-, or multiple-hydrated salt or a mixture thereof.

12. The sustained release solid form of claim 10, wherein the pH-dependent polymer has a dissolution above pH 6.0.

13. The sustained release solid form of claim 10, wherein a ratio of free carboxyl groups to ester groups of the pH-dependent polymer is in a range from about 1:1 to about 1:2.

14. The sustained release solid form of claim 10, wherein the pH-dependent polymer is selected from the group consisting of acrylic acid and methacrylic acid copolymers, methacrylic ester copolymers, ethoxyethyl methacrylate copolymers, methacrylicacylic acid copolymers, amino alkyl methacrylate copolymers, and ammonioalkyl methacrylate copolymers.

15. A method for preparing a sustained release (SR) formulation of opioid receptor antagonists, comprising steps of:
mixing at least one of the opioid receptor antagonist and at least one of pharmaceutical acceptable carrier to form a mixture;
performing a wet granulation on the mixture with a pH-dependent polymer to form sustained release granules;
sieving the sustained release granules through a mesh screen to obtain granules;
compressing the granules to obtain a sustained release (SR) formulation,
wherein the opioid receptor antagonist is selected from the group consisting of Nalmefene, Naltrexone, or a salt thereof.

16. The method of claim 15, further comprises coating the sustained release (SR) formulation with the pH-dependent polymer.

17. The method of claim 15, wherein the opioid receptor antagonist is in the form of an anhydrous salt, a mono-, di-, or multiple-hydrated salt or a mixture thereof.

18. The method of claim 15, wherein the pH-dependent polymer has a dissolution above pH 6.0.

19. The method of claim 15, wherein a ratio of free carboxyl groups to ester groups in the pH-dependent polymer is in a range from about 1:1 to about 1:2

20. The method of claim 15, wherein the H-dependent polymer is selected from the group consisting of acrylic acid and methacrylic acid copolymers, methacrylic ester copolymers, ethoxyethyl methacrylate copolymers, methacrylicacylic acid copolymers, amino alkyl methacrylate copolymers and ammonioalkyl methacrylate copolymers.
